(19) United States
(12) Patent Application Publication
(10) Pub. No.: US 2006/0004243 A1
(43) Pub. Date: Jan. 5, 2006

(54) Radioactive pellet injection apparatus

(75) Inventors: Yasuhiro Shimizu, Kobe-shi (JP); Yoshiki Sawada, Kobe-shi (JP); Makoto Saito, Kobe-shi (JP); Yasuto Shimizu, Kobe-shi (JP); Yasushi Shimizu, Kobe-shi (JP); Yoshitsugu Asada, Kobe-shi (JP)

Correspondence Address: DUANE MORRIS, LLP;IP DEPARTMENT, 30 SOUTH 17TH STREET, PHILADELPHIA, PA, 19103-4196, US

(73) Assignee: SHIMIZUTECH INCORPORATION.

(21) Appl. No.: 11/173,170

(22) Filed: Jul. 1, 2005

(30) Foreign Application Priority Data
Jul. 1, 2004 (JP) 2004-195747

(51) Int. Cl. A61M 36/00

(52) U.S. Cl. 600/7

(57) ABSTRACT

A radioactive pellet injection apparatus includes a console (30) to be disposed beside a bed on which a patient is to be laid. The console (30) and the bed have brackets. An arm (39) is detachably coupled to the bracket of either one of the console (30) and the bed. The arm (39) is movable in either the horizontal or the vertical direction. A rotary driving unit (46) is mounted at a distal end of the arm (39) in such a manner that the tilt angle thereof can be adjusted about a horizontal axis (44) in response to a control signal. The rotary driving unit (46) carries a protective container (48) enclosing a capsule (60) with minute, spherical radioactive pellets enclosed therein, and a radiation detector (49) facing a catheter connecting section (50) extending from the capsule (60). A pump unit (32a) for supplying a carrier fluid for carrying radioactive pellets to the capsule (60) through a tube (51), a control circuit (32b) supplying a control signal to the rotary driving unit (46), and a detector circuit (32c) for processing a detection signal from the radiation detector (49) are disposed in the console (30).

This invention relates essentially to an apparatus for injecting minute, spherical radioactive pellets into a diseased part of a patient for the purpose of treating cancer.

### BACKGROUND OF THE INVENTION

There is an attempt to treat liver cancer, in which a plurality of radioactive minute pellets are injected into a liver artery. The injected radioactive pellets are held in capillaries in the cancer tissue, where they function to cut off nutrition to the cancer tissue and destroy the cancer tissue with the radiation. Radioactive pellets for use in this treatment have a diameter of from about 20 microns to about 30 microns so that they can stay at a location in thick blood vessels upstream the diseased part, and neither can harm sound tissues around the diseased part, nor move to other organs via veins to give side effects to such organs. It is desired that the radioactive pellets should be chemically stable for a long time and not exhibit toxicity. Also, it is desired that the pellets should radiate only a beta ray and have a short half-life period, so that they cannot affect sound tissues. Radioactive pellets which can meet the requirements is of vitreous or ceramic material containing, for example, 90Y (yttrium) having a half-life period of 64 hours or 32P (phosphorus) having a half-life period of 14.3 days. Such spherical minute radioactive pellets may be prepared in the following manner, for example. A required amount of minute spherical pellets of vitreous or ceramic material with yttrium or phosphorus mixed therewith is precisely measured and encapsulated. Then, the capsule is radiated with radiation of neutrons in a nuclear reactor to provide the pellets with radioactivity. The thus prepared minute, spherical radioactive pellets are brought to a medical treatment site, where they are injected into a body of a patient.

In FIG. 1, an apparatus conventionally used for radioactive pellet injection is shown. Radioactive pellets 1 are placed in a phial or medicine bottle 2, which, in turn, is placed in a container 3 made of acryl. The acrylic container 3 is then placed in a container made of lead. The acrylic container 3 is provided with a stopper 7 also made of acryl. The acrylic stopper 7 includes a guide 6 with hypodermic needle guide through-holes 5a and 5b formed therein. A pump unit 8 includes a cylinder 9, a piston 10 and a driver 11 for moving the piston 10. The cylinder 9 is connected to a hypodermic needle 16 via a tube 13, a three-way cock 14, and a tube 15. The needle 16 is inserted into the phial 2.

The three-way cock 14 is also connected via a tube 19 to a container 19, which contains a carrier fluid 18 for carrying radioactive pellets. Another hypodermic needle 20 is inserted into the phial 2 and is connected via a tube 21 to another three-way cock 22, which, in turn, is connected to a catheter 23 and a tube 25 connected to a discharge bottle 24. The discharge bottle 24 is also placed in a lead container 26. Dosimeters indicating a dose are disposed behind the phial 2 and at the proximal end of the catheter 23, respectively.

The amount of residual air in the system including the tubes 13, 15 and 21, the three-way cocks 14 and 22, the phial 2 is very large relative to the amount of radioactive pellets to be injected one time, which is from about 50 mg to about 100 mg. Accordingly, prior to the injection, the residual air must be withdrawn. For that purpose, the tubes 13 and 17 are made to communicate with each other via the three-way cock 14 so that the carrier fluid 18 can be sucked into the cylinder 9. After that, the three-way cock 14 is operated to make the tubes 13 and 15 communicate with each other, and, at the same time, the three-way cock 22 is operated to make the tubes 21 and 25 communicate with each other so that the carrier fluid 18 within the cylinder 9 can be discharged into the bottle 24. This procedure is repeated until the absence of air within the tubes 13, 15 and 21 is confirmed, and, only after that, the tube 21 is changed over to the catheter 23 by means of the three-way cock 22, and the injection is started.

When air is being withdrawn, the carrier fluid 18 must flow through the phial 2, and, therefore, it is inevitable that part of the radioactive pellets 1 is discharged together with the carrier fluid 18. In some cases, a larger amount of the pellets 1 may be undesirably discharged. Also, turbulence of the carrier fluid 18 may occur in the three-way cock 22, which could cause radioactive pellets to adhere to the inner wall of the cock 22, increasing the loss of radioactive pellets. It also involves troublesome cleaning of the cock 22 after the injection. Further, extreme care must be given to the radioactive pellets kept in the discharge bottle 24.

When minute, spherical radioactive pellets are being injected into a diseased part of a patient, fluoroscopy is employed, while adjusting the posture of the patient and the tilt of the phial 2. If the catheter is too short, it could be pulled out during the adjustment.

On the other hand, if the catheter is too long, the radioactive pellets may adhere to the inner surface of the catheter, causing loss of radioactive pellets. Another disadvantage of the above-described apparatus is difficulty of precision control of the flow rate of the radioactive pellets from the phial 2 to the catheter 23.

Therefore an object of the present invention is to realize a radioactive pellet injection apparatus in which air can be discharged easily and in which the flow rate and amount of minute, spherical radioactive pellets to be injected can be precisely controlled.

### SUMMARY OF THE INVENTION

According to the present invention, a cylindrically-shaped capsule of transparent polymeric material is used, which contains minute, spherical radioactive pellets therein. The capsule has stoppers at its opposed ends, each having a hypodermic needle guide through-hole in its center portion. A carrier fluid carrying radioactive pellets to a diseased part of a patient is introduced into the capsule through a hypodermic needle having its distal end inserted into the capsule through the needle guide through-hole at one end thereof. The carrier fluid and the radioactive pellets which are discharged from the capsule through a hypodermic needle inserted into the capsule through the needle guide through-hole at the other end of the capsule are supplied to a catheter, which has its distal end inserted into a diseased part of a patient. In order to prevent radiation from affecting surroundings, the capsule is placed in a protective container made of transparent polymeric material. A first end of the protective container is closed with a stopper having a carrier fluid supply tube connecting portion, to which a carrier fluid supply tube is connected, and a second end is closed with a stopper having a catheter connecting portion, which the catheter is connected to. Each of the two connecting portions of the protective container holds a hypodermic needle to be inserted into the capsule.

The protective container is supported by a rotary driving unit of which a tilt angle can be changed by being rotated about a horizontal axis in response to a control signal. The rotary driving unit supports, in addition to the protective container, a radiation detector facing the catheter connecting section. A pump unit for pumping the carrier fluid for supply to a tube connected to the first end of the protective container, a control circuit which supplies the control signal to the rotary driving unit, and a circuit for detecting the dose based on the detection by the radiation detector, are disposed within a console, which is installed beside a treatment bed on which the patient is to be lain. A supporting arrangement for supporting the rotary driving unit includes a coupling unit, which enables detachable attachment of the supporting arrangement to either the bed or the console.

The capsule and the protective container for the capsule are made of a transparent material so that it can be visually determined how and how much air and radioactive pellets remain in the capsule and the protective container. In view of processability and radiation shielding ability, an acryl resin is a suitable material for the protective container. Physiological salt solution or contrast medium is suitable for use as the carrier fluid.

### BRIEF DESCRIPTION OF THE DRAWINGS

FIG. 1 is a front view of a prior art radioactive pellet injection apparatus.

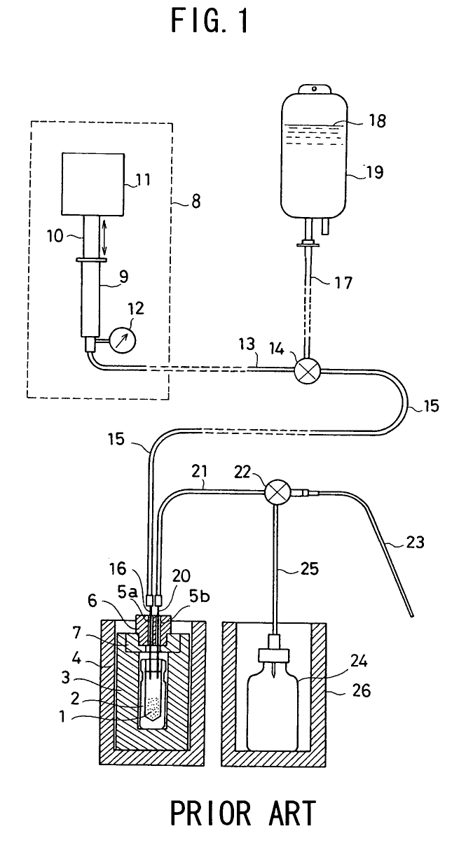

FIG. 2 is a front view of a radioactive pellet injection apparatus according to an embodiment of the present invention.

FIG. 3 is a cross-sectional view of a radioactive pellet capsule in a protective container enclosing the capsule.

### DESCRIPTION OF PREFERRED EMBODIMENT

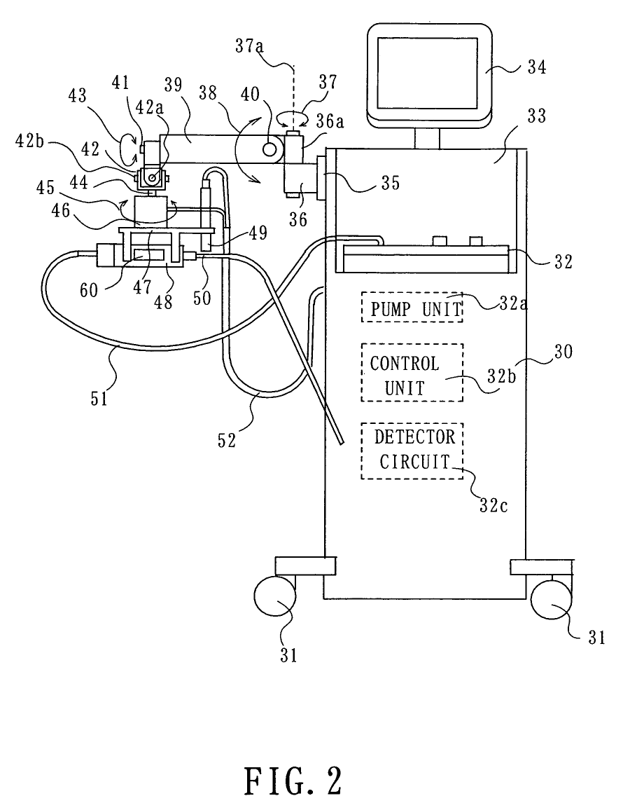

Referring to FIG. 2, a radioactive pellets injection apparatus according to an embodiment of the present invention includes a treatment console 30 having casters 31 attached thereto. On top of the console 30, a control panel 32 is disposed, which includes therein a pump unit 32a, a control circuit 32b and a detector circuit 32c. A rear wall 33 stands upward behind the control panel 32, and a display 34 is disposed on top of the rear wall 33.

A coupler, e.g. a hinge 36, is detachably carried by a bracket 35 on the upper side surface of the console 30. The hinge 36 includes a rotating section 36a rotatably about a vertical center axis 37a as indicated by an arrowed arc 37. An arm 39 is rotatably mounted to the rotating section 36a by means of a spindle 40, which horizontally extends perpendicular to the axis 37a so that the arm 39 can move about the axis 37a and also about the spindle 40 as indicated by an arrowed arc 38. The arm 39 is carried by the rotating section 36a in such a manner that, when it is rotated to a desired position about the axis 37a and the spindle 40, it can be held in that position. One end of a universal joint assembly 42 is mounted to the distal end of the arm 39 by means of a spindle 41 so that it can rotate about the spindle 41 in either direction as indicated by an arrowed arc 43. The manner of mounting the universal joint assembly 42 to the spindle 41 is such that, when the universal joint assembly 42 is rotated to a desired position about the spindle 41, it can be held in that position. The universal joint assembly 42 has a spindle 42a extending perpendicular to the spindle 41 and a spindle 42b extending in parallel with the spindle 41.

The other end of the universal joint assembly 42 is connected via a spindle 44 to a rotary driving unit 46. The rotary driving unit 46 has driving means (not shown) disposed in it and is rotatable about the spindle 44 in either direction as indicated by an arrowed arc 45. A carriage table 47 is mounted to the rotary driving unit 46. The carriage 47 carries a cylindrical, radiation-shielding protective container 48 and a radiation detector 49, which will be described in detail later. A catheter connecting portion 50 is disposed at one end of the protective container 48, to which the radiation detector 49 faces. A tube 51 extends from the other end of the protective container and is connected to the pump unit 32a within the control panel 32. The rotary driving unit 46 and the radiation detector 49 are connected, via a cable 52, to the control circuit 32b and the detector circuit 32c, respectively, within the console 30. The rotary driving unit 46 is arranged to rotate about the spindle 44 in response to a control signal applied thereto from the control circuit 32b.

The rotary driving unit 46 is rotatable also about the spindle 42a of the universal joint assembly 42 and is mounted to the spindle 42a in such a manner that, when it is rotated about the spindle 42a to a desired position, it can be held in that position. Further, the rotary driver 46 is rotatable also about the spindle 42b of the universal joint assembly 42 and is mounted to the spindle 42b in such a manner that, when it is rotated about the spindle 42b to a desired position, it can be held in that position. Accordingly, the rotary driving unit 46 can be rotated about the spindles 42a and 42b of the universal joint assembly 42 to a position in which the spindle 44 of the rotary driving unit 46 is horizontal, and can be held in that position. The rotary driving unit 46 in that position can be further rotated about the spindle 44 so that the tilt angles the protective container 48 and the radiation detector 49 with respect to a horizontal plane can be changed. The arm 39, the universal joint assembly 42, the rotary driving unit 46 and the carrying table 47 form together a supporting arrangement.

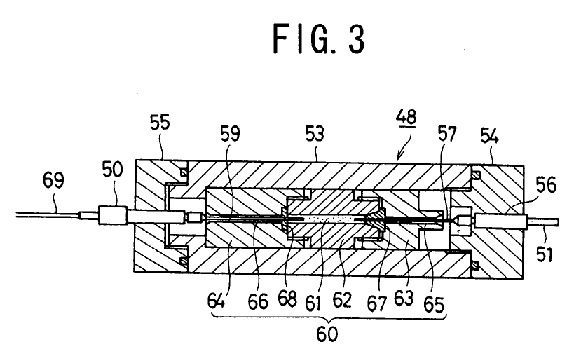

As shown in FIG. 3, the protective container 48 is formed of a cylindrical member 53 enclosing a capsule 60, and stoppers 54 and 55 screwed into the opposed ends of the cylindrical member 53. The cylindrical member 53 and the stoppers 54 and 55 are formed of a transparent polymeric material, e.g. an acrylic resin, and their walls are thick. The stopper 54 has a tube connecting section 56 to which the tube 51 connected to the pump unit 30a is connected. A hypodermic needle 57 extending into the interior of the cylindrical member 53 is attached to the tube connecting section 56. The stopper 55 has a catheter connecting section 50 extending outward of the cylindrical member 53. A hypodermic needle 59 extending into the interior of the cylindrical member 53 is attached to the catheter connecting section 50.

The capsule 60 includes a main body 62 having a cavity 61 in which minute, spherical radioactive pellets are enclosed, and stoppers 63 and 64 screwed into the opposite ends of the main body 62. The main body 62 and the stoppers 63 and 64 are formed of a transparent polymeric resin, e.g. an acrylic resin. The stoppers 63 and 64 have needle guide through-holes 65 and 66, respectively. Rubber packing members 67 and 68 are interposed between the cavity 61 and the needle guide through-holes 65 and 66, respectively.

Thus, when the stoppers 54 and 55 are screwed into the opposite ends of the protective container 48 with the capsule 60 placed therein, the needles 57 and 59 extends into the cavity 61 of the capsule 60 through the needle guide through-holes 65 and 66, respectively, whereby a path is formed to extend from the tube connecting section 56, through the needle 57, the cavity 61 and the needle 59 to the catheter connecting section 50.

For providing medical treatment with radioactive pellets, a dummy capsule (not shown) with only the carrier fluid contained in the protective container 48, but without radioactive pellets, is loaded. Then, the pump unit 32a is operated, with the catheter connecting section 50 opened, to thereby push out the air in the system from the pump unit 32a to the catheter connecting section 50 completely through the catheter connecting section 50, while monitoring the capsule 60. At the same time, the catheter 69 is inserted into a diseased part of a patient to be treated, while looking at the catheter 69 through, for example, a fluoroscope.

After the air within the tube 51 is completely discharged, a normal capsule 60 having its cavity 61 filled with radioactive pellets and carrier fluid is substituted for the dummy capsule in the protective container 48. With this apparatus, since the system from which air has to be removed includes only the pump unit 32a and the path extending from the pump unit 32a to the hypodermic needle 57 inserted into the capsule 60 and does not include radioactive pellets, operation to remove air is easy.

Thereafter, the hinge 36 with the arm 39 mounted thereto is detached from the bracket 35 on the console 30, and, then, attached to a bracket (not shown) mounted on a side of the medical treatment bed on which the patient is lying. Then, the proximal end of the catheter 69 having its distal end inserted into the diseased part of the patient is connected to the catheter connecting section 50 of the protective container 48.

After that, the protective container 48 is brought to a position near to the patient by rotating the arm 39 about the rotating section 36a and the spindle 40 and rotating the universal joint assembly 42 about the spindle 41. Then, the rotary driving unit 46 is rotated about the spindle 42a and/or the spindle 42b of the universal joint assembly 42 to adjust the position of the rotary driving unit 46 so that the spindle 44 of the rotary driving unit 46 assumes a horizontal position. Then, the pump unit 32a is activated to inject the radioactive pellets in the carrier fluid in the capsule 60 into the diseased part through the catheter 69. Since the capsule 60 containing radioactive pellets is fixed indirectly to the treatment bed during the injection, the catheter is prevented from falling out from the diseased part of the patient if the bed moves or tilts. Furthermore, since the distance between the capsule 60 and the diseased part is short, with no components except the catheter 69 intervening, loss of radioactive pellets in the injection path can be minimized.

The amount of radioactive pellets injected can be observed from outside the protective container 48. In addition, the detector circuit 32c processes an output signal from the radiation detector 49 and causes the amount injected and the injection rate to be displayed on the display 34. The rate of injection can be adjusted by adjusting the rate at which the carrier fluid is pumped by the pump unit 32a. The tilting of the protective container 48 can be adjusted by rotating the rotary driving unit 46 about the spindle 44 in response to a control signal provided by the control circuit 32b, whereby the injection rate can be adjusted. In other words, it is possible to control the injection rate to be constant, which also can be displayed on the display 34.

It is desirable that the dummy capsule be made of colored material so that it can be readily discerned from a normal capsule containing radioactive pellets. Preferably, the injection apparatus with a dummy capsule placed in a protective container should be sent to a treatment site, together with a normal capsule filled with radioactive pellets and carrier fluid attached separately.

What is claimed is:

1. A radioactive pellet injection apparatus comprising:
a capsule made of transparent polymeric material, including a cylindrical capsule main body having a cavity in a center portion thereof, and a pair of stoppers attached to respective ones of opposing ends of said capsule main body, each of said stoppers having a through-hole at a center thereof extending into said cavity for guiding a hypodermic needle into said cavity, said cavity containing minute, spherical radioactive pellets therein;
a protective container made of transparent polymeric material for enclosing said capsule, having one end thereof closed by a stopper having a carrier fluid supply tube connecting section on which one of said hypodermic needles is mounted to extend into said capsule cavity, and having the other end thereof closed by a stopper having a catheter connecting section on which the other of said hypodermic needles is mounted to extend into said cavity;
a radiation detector disposed to face a portion of said catheter connecting section outside said protective container;
a rotary driving unit for supporting said protective container and said radiation detector, a tilt angle of said rotary driving unit being variable about a horizontal axis in response to a control signal;
a console including a pump unit for supplying a radioactive pellet carrier fluid to a tube connected to said carrier fluid supply tube connecting section, a detector circuit for processing a detection signal from said radiation detector, and a control circuit providing the control signal to said rotary driving unit;
and a supporting arrangement having a coupling unit at a proximal end thereof, said coupling unit being capable of being detachably coupled to either of said console and a treatment bed on which a patient is to be laid, said rotary driving unit being disposed at a distal end of said supporting arrangement.

2. The apparatus according to claim 1 wherein said rotary driving unit is disposed at said distal end of said supporting arrangement in such a manner that said rotary driving unit can rotate about two axes perpendicular to a rotation axis of the rotary driving unit.

3. The apparatus according to claim 1 wherein said supporting arrangement includes an arm rotatable about vertical and horizontal axes relative to said coupling unit.

4. The apparatus according to claim 3 wherein said rotary driving unit is mounted to a distal end of said arm in such a manner that said rotary driving unit can rotate about two axes extending perpendicular to a rotation axis thereof.
